(19) United States
(12) Patent Application Publication
(10) Pub. No.: US 2020/0058386 A1
(43) Pub. Date: Feb. 20, 2020

(54) ADAPTIVE ARTIFICIAL INTELLEGENCE INSULIN PUMP

(71) Applicant: Samatha Grace Kelly, Western Springs, IL (US)

(72) Inventor: Samatha Grace Kelly, Western Springs, IL (US)

(21) Appl. No.: 16/594,056

(22) Filed: Oct. 6, 2019

(51) Int. Cl. G16H 20/17; G06N 5/04; A61M 5/172; A61M 39/02; A61M 5/14

(52) U.S. Cl. CPC G16H 20/17; G06N 5/04; A61M 5/1723; A61M 39/02; A61M 2205/502; A61M 2230/201; A61M 2039/0205; A61M 2230/63; A61M 2205/3584; A61M 5/1408

(57) ABSTRACT

An artificial intelligence insulin pump that processes glucose data, wellness data, external data, and port information to determine the appropriate amount and timing of insulin to deliver to a person. External data includes data from sources remote from the insulin pump and the user such as social media data, GPS location data, historical data including energy and food intake at locations visited prior, data from third party sources. Such external data may include that the use is registered for a marathon and in at the marathon venue and the user's exertion level indicates that the user is running has begun to run the marathon. The AI engine would than adjust the insulin appropriately.

### BACKGROUND

Diabetes affects many people. Type 1, also called juvenile diabetes, diabetes is partially difficult for a child to manage. Artificial insulin is delivered via needle injections or through an insulin pump attached to the body through a port. Existing insulin pumps include the Medtronic MiniMed 630G, 640G and 670G; Animas Corp.'s OneTouch Ping and Vibe; Insulet Corp.'s Omnipod; and Sooil Development's Dana Diabecare IIS; and Tandem Diabetes Care's T:flex Pump.

However, traditional dosing of insulin by needles or a pump is limited by the information utilized to determine the dose and timing. Incorrect dosing can cause high or low blood glucose levels and in extreme cases death. Some insulin pumps are vulnerable to hacking allowing the hacker to control the dosing.

### BRIEF DESCRIPTION OF THE DRAWINGS

FIG. 1 depicts an adaptive artificial intelligence insulin pump for implementation and/or support of according to some embodiments.

FIG. 2 depicts an adaptive artificial intelligence insulin pump for implementation and/or support of according to some embodiments.

FIG. 3 depicts a flow diagram of the process according to some embodiments.

### DETAILED DESCRIPTION

The new and improved insulin pump utilizes additional information and artificial intelligence to provide diabetics with improved insulin delivery.

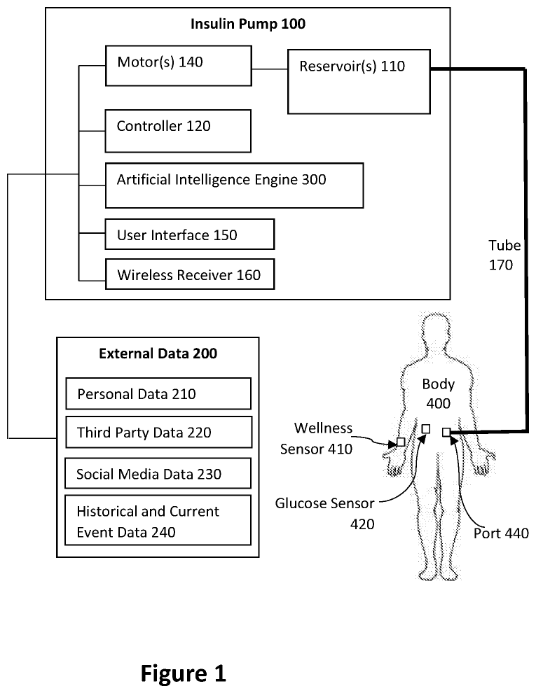

Referring to FIG. 1, an insulin pump 100 can receive input manually from the user through a user interface 150. The user can enter an amount of insulin to be delivered, a carbohydrate count and/or a blood glucose level that the insulin pump 100 uses to determine the amount of insulin to be delivered to the body 400 through the port 440. Further, the insulin pump 100 can deliver a basal amount of insulin throughout the day. The basal amount can be preset levels that change based on time of day or they can be set by the artificial intelligence engine 300. When a bolus dose of insulin is determined to be needed the artificial intelligence engine can use multiple data sources to determine the optimal timing and dosing of the bolus dosage. Such data sources can include the data entered in the user interface 150, the external data 200, and the data from the body 400. For example, the user may estimate that the body 400 is about to consume 100 carbohydrates in a meal and the user enters that into the user interface 150. Then the artificial intelligence engine 300 adaptively adjusts the normally recommended dose by recognizing that this user has typically entered the carbohydrates after the body 400 had consumed the carbohydrates rather than before. A faster acting insulin may be delivered, or the normal insulin may be delivered sooner. Further, the AI engine 300 may determine that the carbohydrate data was enter much after the meal was consumed by the body 400 in part by recognizing the time stamp on the credit card of the user that indicates when the meal was purchases, the type of meal (fast food, formal dining, concert setting) in addition to the use of other external data this be detecting that the body 400.

Additional information would make such manual enter better or eliminate the need for that manual input. A Glucose Sensor 420 can measure the blood glucose or other glucose level from the body 400 and communicate that information to the insulin pump 100. The communication from the glucose monitor and other devices can be wired or wireless, including using Bluetooth or another wireless communication mechanism. The Glucose Sensor 420 reduces or eliminate the user's need to manually test the glucose level.

In various embodiments of the inventions, an artificial intelligence engine 300, a wellness sensor 410, and external data 200 may be used various combination to improve the determination of the amount and timing of the delivery of the insulin.

Further, in an embodiment the insulin pump 100 may include multiple motors 140 and multiple reservoirs 110 to deliver different types of insulin (e.g. slow acting and fast acting), glucose, glucagon, vitamins, supplements, and other medications.

The Glucose Sensor 420 may be a simple monitor such as MiniMed model MMT-7008A Enlite Continuous Blood Glucose Sensor or a more advanced sensor. The sensor may detect blood glucose directly as the Enlite does or indirectly. Such indirect sensing may include sensing glucose levels without penetrating the skin or even sensors positioned around a room, such as a dinning room, bedroom, a restaurant, and remote from the body 400.

The wellness sensor 410 may be a simple fitness watch like a Fitbit (e.g. Charge 3) or a Garmin (e.g. VivoSmart 4) or a more complex sensor. The more complex sensor may include the ability to detect sickness, sleep patterns, anxiety, pulse rate, medication in the blood stream or body, injury or blood loss, smoking or use of tobacco and nicotine products and other prescription and nonprescription drugs including illegal drugs.

The external data 200 includes information from external sources but can also include data from the insulin pump 100, the Glucose Sensor 420, the wellness sensor 410, and the port 500. The external sources may include social media tools and websites, medical information, prescription/pharmacy information, calendars (work calendar, personal calendar, national holiday calendar, school calendar, etc.), global positioning systems (GPS), credit/debit card information, sick day notification from school or work, payday/welfare payment/gifts notifications. The age of the port 500 may be sent from the port to the pump 100, from the port to the AI engine 300, from data manually input into the pump 100, or from information external to the body 400 and the pump 100.

The artificial intelligence engine 300, also called the AI engine, may be incorporated into the insulin pump 100 or it may be remote from the insulin pump 100 and the body 400. The AI engine 300 receives data from the some of the following: external data 200, the insulin pump 100, and the sensors on the body 400 including the Glucose Sensor 420, the wellness sensor 410, and the port 500.

The AI engine 300 can monitor the actual dosing of insulin provided by the insulin pump 100 to the body 400 and determine if past dosing was appropriate and if a future dosing is appropriate. The AI engine 300 does this not by merely setting limits on dosing per hour or maximum/minimum bolus or basal dosing but by interpreting the body's 900 past response to insulin dosing along with the external data 200 and the data from the sensors 300, 400, 500 on the body 400.

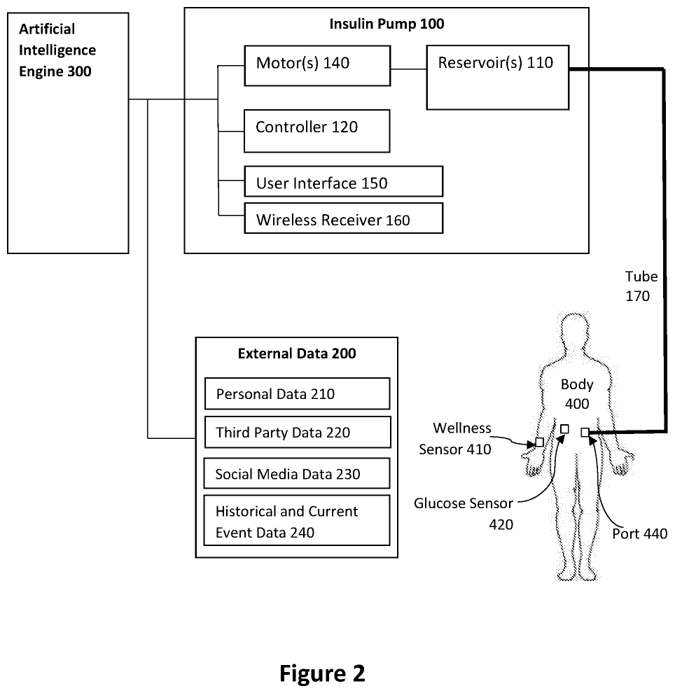

Referring to FIG. 2, an artificial intelligence engine 300 is shown remote from the insulin pump 100. The artificial intelligence engine 300 may access the remote data 200 and the data from the body 400 including data from the wellness sensor 410, the Glucose Sensor 420 and the port 500.

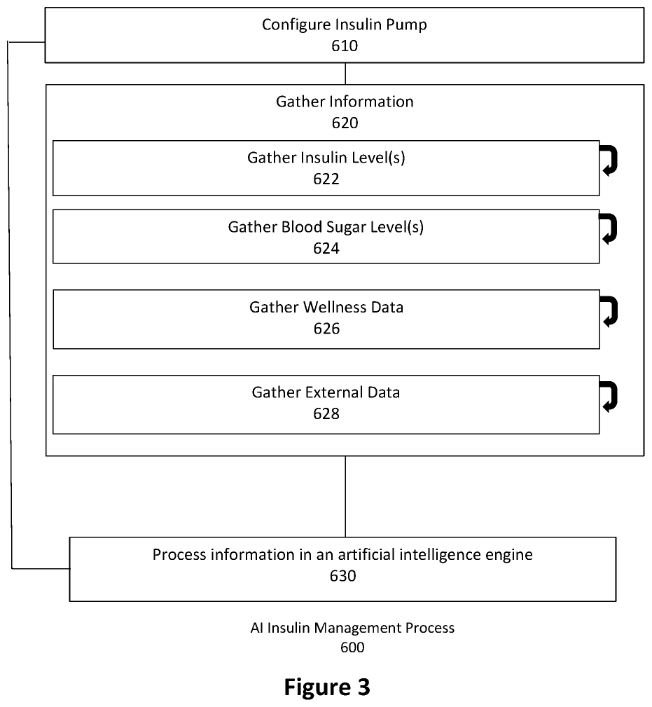

FIG. 3 demonstrates an AI insulin management process 600 using an artificial intelligence engine 300 to deliver insulin from an insulin pump 100 to a body 400 through a tube 170 to a port 440. The steps of the AI Insulin Management Process 600 are typically done iteratively, in parallel, and independently. The steps can be sequenced or timed to save processing power or battery life.

Step 610 includes configuring the insulin pump. This step may only need to be done when the insulin pump 100 is initially set up or when the AI engine 300 needs to be resynced with the insulin pump 100. Alternatively, the insulin pump 100 may need periodic calibrations and step 610 may need to be executed more frequently.

Step 620 includes gathering various types of information and sharing that information with the insulin pump and/or the AI engine 300. Some of the information gathered in step 620 includes the insulin level(s) (multiple levels over time, from different monitors, using different data interpretation techniques) in step 622, blood sugar level(s) (multiple levels over time in step 624, from different monitors, using different data interpretation techniques), wellness data, including heart rate data, activities data, respiration data, mental health data, alcohol level data, drug levels data in step 626, and external data in step 628.

Step 630 process the information gathered in step 620 through an artificial intelligence engine 300 and supplies input to the insulin pump 100 in step 610. This process 600 is repeated on a near continuous basis in real-time.

By using the artificial intelligence insulin management process 600 the insulin pump 100 provides better delivery of insulin from the insulin pump 100 to the body 400.

The illustrations of the embodiments described herein are intended to provide a general understanding of the structure of the various embodiments. The illustrations are not intended to serve as a complete description of all of the elements and features of apparatus and systems that utilize the structures or methods described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

While this specification contains many specifics, these should not be construed as limitations on the scope of the invention or of what may be claimed, but rather as descriptions of features specific to particular embodiments of the invention. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings and described herein in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the described embodiments should not be understood as requiring such separation in all embodiments, and it should be understood that the described program components and systems can generally be integrated together in a single software product or packaged into multiple software products.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

It is therefore intended that the foregoing detailed description be regarded as illustrative rather than limiting, and that it be understood that it is the following claims, including all equivalents, that are intended to define the spirit and scope of this invention.

1-34. (canceled)

35. A method for delivering medication, comprising:
receiving glucose data from a glucose sensor near a body;
receiving wellness data from a wellness sensor near the body;
receiving external data from away from the body;
determining a need for insulin or glucose using an artificial intelligence engine utilizing the glucose data, wellness data and external data; and
administrating to a body from an insulin pump a dose of insulin or glucose based on the determined need.

36. The method of claim 35 wherein the administrating including selecting from a plurality of reservoirs having multiple types of insulin.

37. The method of claim 35 wherein the administrating including selecting from a plurality of reservoirs having multiple types of glucose including glucagon.

38. The method of claim 35 wherein the receiving external data includes receiving data from at least one of the following sources associated with the body: a social media account, global positioning system information, information indicating the past location, information indicating the present location, information on speed, credit card transactions, debit card transactions, calendar entry including school calendars, government calendars, and personal calendars, attendance information from a school or an employer, medication and refill information from a pharmacy, and insulin pump information from a second insulin pump.

39. The method of claim 35 wherein the determining includes determining the age of a port used to administer insulin to the body.

40. An insulin pump comprising:
a controller for controlling the delivery of insulin,
a reservoir containing insulin connected to the controller,
a port attached to a body connected to the reservoir by a tube, and
an artificial intelligence engine,
wherein the artificial intelligence engine determines an amount of insulin to deliver based on external data received from a location remote from the body,
wherein the artificial intelligence engine transmits the amount of insulin to the controller.

41. The insulin pump claim 40, wherein the receiving external data includes receiving data from at least one of the following sources associated with the body: a social media account, global positioning system information, information indicating the past location, information indicating the present location, information on speed, credit card transactions, debit card transactions, calendar entry including school calendars, government calendars, and personal calendars, attendance information from a school or an employer, medication and refill information from a pharmacy, and insulin pump information from a second insulin pump.

42. The insulin pump claim 41, wherein the external data includes historical and current data.

43. The insulin pump claim 41, wherein the artificial intelligence engine is remote from the insulin pump.

44. The insulin pump claim 41, wherein the artificial intelligence engine recognizes a pattern of spending on a credit card or debit card as it related to food intake into the body.

45. The insulin pump claim 41, wherein the artificial intelligence engine recognizes pattern of the age of the port and the effectiveness of the insulin on the body.

46. The insulin pump claim 41, wherein the artificial intelligence engine recognizes periodic patterns of behavior including exercise and diet.

47. The insulin pump claim 41, wherein the artificial intelligence engine adapts the amount of insulin based a recognition of the impact of medication information on the body.

48. The insulin pump claim 41, wherein insulin pump includes multiple reservoirs containing different types of insulin.

49. The insulin pump claim 41, wherein insulin pump includes multiple reservoirs containing insulin and at least one of the following: glucose or glucagon.

50. An artificial intelligence engine for determining the amount of insulin to deliver to a body via an insulin pump, comprising:
a first receiver for receiving external information from a location remote from the body, wellness information from the body, and glucose information from the body;
a storage unit to store historical information including external information from remote from the body, wellness information from the body, and glucose information from the body and the body's response to past dosages on insulin in various circumstances and data manually entered into the insulin pump; and
a transmitter for transmitting insulin information to the insulin pump.

51. The artificial intelligence engine of claim 50, wherein the external data includes data from at least one of the following sources associated with the body:
a social media account,
global positioning system information,
information indicating the past location,
information indicating the present location,
information on speed,
credit card transactions,
debit card transactions,
calendar entry including school calendars, government calendars, and personal calendars,
attendance information from a school or an employer,
medication and refill information from a pharmacy, and
insulin pump information from a second insulin pump.

52. The artificial intelligence engine of claim 50, wherein artificial intelligence engine provides the insulin pump information for multiple reservoirs containing different types of insulin.

53. The artificial intelligence engine of claim 50, wherein artificial intelligence engine provides the insulin pump information for multiple reservoirs containing insulin and at least one of the following: glucose or glucagon.

54. The artificial intelligence engine of claim 50, wherein the artificial intelligence engine utilized data manually entered in an insulin pump user interface in addition to remote data.
